(19) United States
(12) Patent Application Publication
(10) Pub. No.: US 2007/0178480 A1
(43) Pub. Date: Aug. 2, 2007

(54) Extended dynamic range reading of chemical arrays

(76) Inventors: John F. Corson, Mountain View, CA (US); Bo Curry, Redwood City, CA (US); Charles Troup, Livermore, CA (US); Xiangyang Zhou, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/344,773

(22) Filed: Jan. 31, 2006

(51) Int. Cl. C12Q 1/68; G06F 19/00

(52) U.S. Cl. 435/6; 702/20

(57) ABSTRACT

Methods of reading chemical arrays are provided. Aspects of the methods include reading an array at a first and second detector gain setting to produce linked data sets of an array, where each reading may be made using a different detector gain setting. Aspects of the methods further include extracting features from the linked data sets, where in certain embodiments a merged data set feature extraction protocol is employed. Aspects of the invention further include programming for operating devices, e.g., chemical array readers, as well as readers comprising such programming.

### BACKGROUND

Arrays of surface-bound binding agents, i.e., chemical arrays, may be used to detect the presence of particular targets, e.g., biopolymers like polypeptides and nucleic acids, in solution. The surface-bound binding agents (i.e., probes) may be oligonucleotides, peptides, polypeptides, proteins, antibodies or other molecules capable of binding with target molecules in solution. Such binding interactions are the basis for many of the methods and devices used in a variety of different fields, e.g., genomics (in sequencing by hybridization, SNP detection, differential gene expression analysis, identification of novel genes, gene mapping, finger printing, etc.), CGH, location analysis and proteomics.

One type of array assay method uses probe molecules immobilized in an array pattern of features on a surface of a first solid support, such as a glass slide. A fluid containing sample is contacted with the surface, covered with another solid support such as a coverslip to form an assay area and then placed in an environmentally controlled chamber, such as an incubator. The targets in the sample bind to the complementary-probes of the array of the first solid support to form binding complexes on the array. In certain instances, the target molecules are labeled with a detectable label, such as a fluorescent label or chemiluminescent label. The resultant binding complexes on the array are then detected and read, for example by optical means. Laser light may be used to excite fluorescent tags, generating a signal only in those features (e.g., in the form of spots) of the array that have a target molecule and thus a fluorescent label bound to a probe molecule. This pattern may then be read, e.g., digitally scanned for computer analysis. The pattern of binding by target molecules to probe features on the solid support surface provides desired information about the sample, e.g., the determination of the presence of one or more analytes of interest in the sample.

Current detection methodologies, however, are limited because the range of light intensity emitted by an array generally exceeds the linear dynamic range of the photodetection systems used for the detection of that light. Accordingly, in scanning an array, photodetection systems may produce a significant number of data points that are either saturated (i.e., at or above the maximum of the linear dynamic range of the detector), or indistinguishable from background (i.e., at or below the minimum of the dynamic range of the detector).

### SUMMARY

Methods of reading chemical arrays are provided. Aspects of the methods include reading an array at a first and second detector gain setting to produce first and second data sets, e.g., in the form of first and second images, where each reading may be made using a different detector gain setting. Aspects of the methods further include extracting features from the produced data sets, where in certain embodiments a merged image feature extraction protocol is employed. Aspects of the invention further include programming for operating devices, e.g., chemical array readers, as well as chemical array readers that include such programming.

Aspects of the invention include methods of reading a chemical array, where the methods comprise: positioning the array in a reading position of a chemical array reader; reading the array a first time in the scan position at a first detector gain setting to produce a first data set; and before moving the array out of the reading position re-reading the array at a second detector gain setting to produce a second data set. In certain embodiments, the re-reading occurs immediately after said reading of said array a first time. In certain embodiments, the method further comprises selecting whether to read the array in an extended dynamic range mode. In certain embodiments, when a choice is made to read the array in an extended dynamic range mode, the second detector gain setting is less than the first detector gain setting. In certain embodiments, the method further comprises selecting the first and second detector gain settings. In certain embodiments, the method further comprises linking said first and second data sets, e.g., with an identifier, such as a name identifier. In certain embodiments, the method further comprises feature extraction from the first and second scan data sets, e.g., using a protocol that comprises merging the first and second data sets into a merged data set.

Aspects of the invention further include methods of extracting features from two or more linked data sets of a chemical array, e.g., as produced above. Such methods may include determining whether the linked data sets should be merged or used separately; and, if a decision is made to merge the linked data sets, converting signals in the linked data sets to background subtracted signals; and combining the background-subtracted signals into a merged set. In certain embodiments, prior to the combining step the method further comprises correcting background subtracted signals to account for differences in detector gain, e.g., by adjusting the background subtracted signals from a second of said linked data sets by a detector gain difference correction factor. In certain embodiments, the detector gain difference correction factor is determined by: defining an acceptable signal range; and determining the median of the ratios of the signals from the linked data sets that fall within the acceptable signal range to determine the detector gain difference correction factor. In certain embodiments, the combining comprises selecting data from a given set based on whether a signal in a first set exceeds a threshold. In certain embodiments, the combining comprises producing a merged set of signals in which signals from saturated features from a first of said linked data sets are substituted by signals from corresponding features from a second of said linked data sets. In certain embodiments, the combining comprises averaging background subtracted signals from said linked data sets.

Aspects of the invention further include computer-readable media encoding instructions to direct a processor, e.g., present in chemical array reader or computer, to perform the extended dynamic range reading and/or feature extraction methods, such as described above.

Aspects of the invention further include methods of assaying a sample, e.g., by contacting the sample with a chemical array of two or more ligands immobilized on a surface of a solid support at different known locations; and reading the array with a chemical array reader according to obtain two or more linked images of the array. In certain embodiments, the method further comprises extracting features from the two or more linked images, e.g., using the feature extraction protocols of the invention. In certain embodiments, the chemical array is chosen from a polypeptide array and a nucleic acid array.

### BRIEF DESCRIPTION OF THE DRAWINGS

FIG. 1 illustrates a substrate carrying an array, such as may be fabricated by methods of the present invention;

FIG. 2 is an enlarged view of a portion of FIG. 1 showing multiple spots or features;

FIG. 3 is an enlarged illustration of a portion of the substrate in FIG. 2;

FIG. 4 provides a flow chart of an array reading process according to an embodiment of the invention;

FIG. 5 provides a flow chart of a feature extraction process according to an embodiment of the invention;

FIG. 6 provides a flow chart of an XDR array reading and feature extraction protocol according to an embodiment of the invention;

FIG. 7 schematically illustrates an embodiment of an optical reader system of the present invention;

FIG. 8 provides a graphical representation of the ratio of signal from high gain to low gain scan according to an embodiment of the invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid. A “biomonomer” references a single unit, which can be linked with the same or other biomonomers to form a biopolymer (e.g., a single amino acid or nucleotide with two linking groups, one or both of which may have removable protecting groups).

The terms “nucleoside” and “nucleotide” are intended to include those moieties which contain not only the known purine and pyrimidine bases, but also other heterocyclic bases that have been modified. Such modifications Include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

A “biopolymer” is a polymer of one or more types of repeating units. Biopolymers are typically found in biological systems (although they may be made synthetically) and may include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” may include DNA (including cDNA), RNA, oligonucleotides, and PNA and other polynucleotides as described in U.S. Pat. No. 5,948,902 and references cited therein (all of which are incorporated herein by reference), regardless of the source.

The term “biomolecule” means any organic or biochemical molecule, group or species of interest that may be formed in an array on a substrate surface. Exemplary biomolecules include peptides, proteins, amino acids and nucleic acids.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate.

The term “sample” as used herein relates to a material or mixture of materials, typically, although not necessarily, in fluid form, containing one or more components of interest.

A biomonomer fluid or biopolymer fluid reference a liquid containing either a biomonomer or biopolymer, respectively (typically in solution).

The term “peptide” as used herein refers to any polymer compound produced by amide formation between an α-carboxyl group of one amino acid and an α-amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 up to about 200 nucleotides in length, e.g., from about 25 to about 200 nt, including from about 50 to about 175 nt, e.g. 150 nt in length

The term “polynucleotide” as used herein refers to single- or double-stranded polymers composed of nucleotide monomers of generally greater than about 100 nucleotides in length.

An “array,” or “chemical array” used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. As such, an addressable array includes any one or two or even three-dimensional arrangement of discrete regions (or “features”) bearing particular biopolymer moieties (for example, different polynucleotide sequences) associated with that region and positioned at particular predetermined locations on the substrate (each such location being an “address”). These regions may or may not be separated by intervening spaces. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm² For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

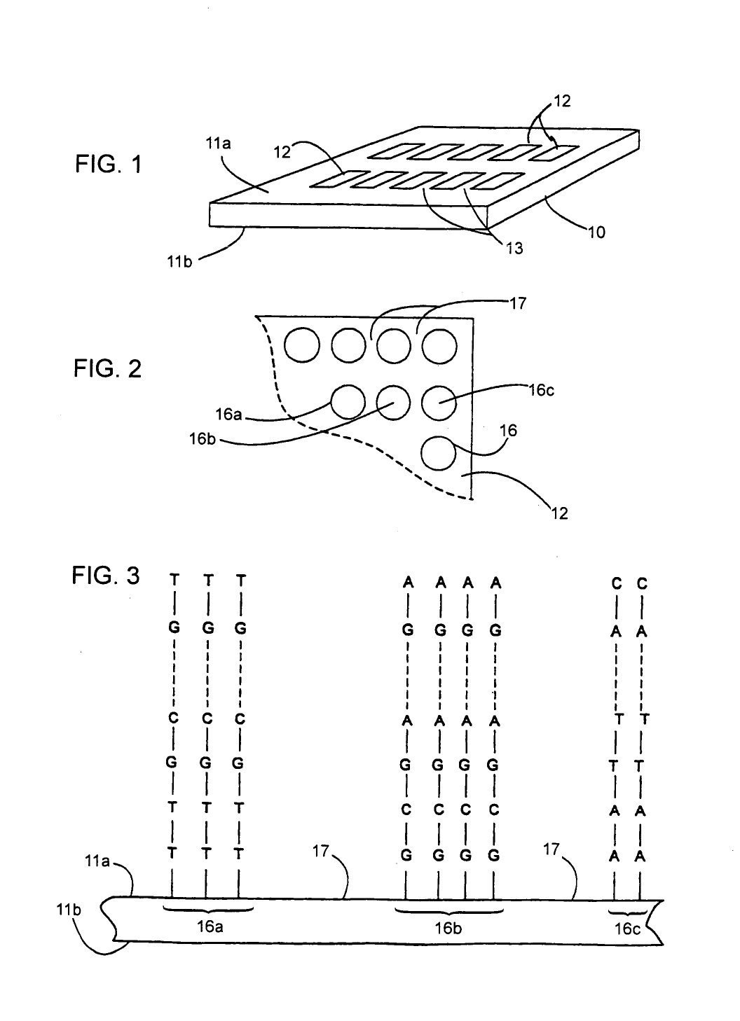

An exemplary chemical array is shown in FIGS. 1-3, where the array shown in this representative embodiment includes a contiguous planar substrate 10 carrying an arrays 12 disposed on a surface 11a of substrate 10. It will be appreciated that more than one array (any of which are the same or different) may be present on surface 11a, with or without spacing between such arrays. That is, any given substrate may carry one, two, four or more arrays disposed on a front surface of the substrate and depending on the use of the array, any or all of the arrays may be the same or different from one another and each may contain multiple spots or features. Each array 12 can be separated by an inter-array domain 13. A second surface 11b of the slide 10 does not carry any arrays 12. Each array 12 can be designed for testing against any type of sample, whether a trial sample, reference sample, a combination of them, or a known mixture of biopolymers such as polynucleotides. Substrate 10 may be of any shape, as mentioned above. The substrate may be porous or non-porous. The substrate may have a planar or non-planar surface.

As mentioned above, array 12 contains multiple spots or features 16 of biopolymer ligands, e.g., in the form of polynucleotides. As mentioned above, all of the features 16 may be different, or some or all could be the same. The interfeature areas 17 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 11a and the first nucleotide.

Substrate 10 may carry on surface 11a, an identification code, e.g., in the form of bar code (not shown) or the like printed on a substrate in the form of a paper label attached by adhesive or any convenient means. The identification code contains information relating to array 12, where such information may include, but is not limited to, an identification of array 12, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.133 SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position, place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

A “scan region” refers to a contiguous (such as rectangular) area in which the array spots or features of interest are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that many computer-based systems are available which are suitable for use in the present invention. The data storage means may comprise any manufacture comprising a recording of the present information as described above, or a memory access means that can access such a manufacture.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Computer readable medium” as used herein refers to any storage or transmission medium that participates in providing instructions and/or data to a computer for execution and/or processing. Examples of storage media include floppy disks, magnetic tape, UBS, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. A file may be stored in permanent memory.

With respect to computer readable media, “permanent memory” refers to memory that is permanently stored on a data storage medium. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “memory” or “memory unit” refers to any device which can store information for subsequent retrieval by a processor, and may include magnetic or optical devices (such as a hard disk, floppy disk, CD, or DVD), or solid state memory devices (such as volatile or non-volatile PAM). A memory or memory unit may have more than one physical memory device of the same or different types (for example, a memory may have multiple memory devices such as multiple hard drives or multiple solid state memory devices or some combination of hard drives and solid state memory devices).

Items of data are “linked” to one another in a memory when the same data input (for example, filename or directory name or search term) retrieves the linked items (in a same file or not) or an input of one or more of the linked items retrieves one or more of the others.

A “reader” or “scanner” is a device for evaluating arrays. In certain readers, an optical light source, such as a laser light source, generates a collimated beam. The collimated beam is focused on the array and sequentially illuminates small surface regions of known location (i.e., a position) on an array substrate. The resulting signals from the surface regions are collected, e.g., by gathering the array image on a point-by-point basis or via an imaging system g(collecting signals from an extended area on the array). In certain embodiments, a confocal system is employed. Where desired, the collected signals are filtered using an appropriate dichroic filter, or set of filters, to discriminate between fluorescence from the dye molecules and light at the excitation wavelength. A recording device, such as a computer memory, records the detected signals and builds up a raster scan file of intensities as a function of position, or time as it relates to the position. Such intensities, as a function of position, are referred to herein as “pixels”. Arrays may be scanned and/or scan results may be represented at 2-50 micron pixel resolution, and such as at 2-20 micron resolution, and including at 2-10 micron spatial resolution. To achieve the precision required for such activity, components such as the lasers may be set and maintained with particular alignment. Scanners may be bidirectional, or unidirectional, as is known in the art. A reader typically used for the evaluation of arrays includes a scanning fluorometer. A number of different types of such devices are commercially available from different sources, such as Perkin-Elmer, Agilent, or Axon Instruments, etc., and examples of typical scanners are described in U.S. Pat. Nos: 5,091,652; 5,760,951, 6,320,196 and 6,355,934.

The terms “assessing” and “evaluating” are used interchangeably to refer to any form of measurement, and include determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent. The term “evaluating a pixel” and grammatical equivalents thereof, are used to refer to measuring the strength, e.g., magnitude, of pixel signal to determine the brightness of a corresponding area present on the surface of an object scanned.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station. In certain embodiments, a processor may be a “signal processor”, where a signal processor receives input signals and processes those signals. A signal processor may be programmed or hard wired to perform one or more mathematical functions, such as those described below.

In certain embodiments, a signal processor may “integrate” a set of digital signals (e.g., a set of digital signals representing an analog signal or a digitized version of an analog signal) to produce an integrated data signal. By “integrating” is meant that a set of digital signals is input into a signal processor and the signal processor provides an output signal, where in certain embodiments a single output signal represents the set of input signals. In certain embodiments, the input set of digital signals may be integrated by summing the set of input signals. If an analog signal is referred to as being integrated, then it is understood that the analog signal is first digitized (i.e., sampled) prior to integration. For example, if an analog signal for a pixel is to be integrated, the signal is first sampled and digitized to provide a set of digital signals, and those digital signals are integrated by a signal processor to provide an output signal, typically a binary signal that represents a numerical evaluation of the overall magnitude of the input set of digital signals (thereby providing a numerical evaluation of the magnitude of the analog signal for the pixel). The output of a signal processor may be referred herein as “data” and may be stored in memory.

Data from reading an array may be raw data (such as fluorescence intensity readings for each feature in one or more color channels, or for example, the output of a signal processor that has integrated a set of digital signals for a pixel) or may be processed data such as obtained by rejecting a reading for a feature which is below a predetermined threshold and/or forming conclusions based on the pattern read from the array (such as whether or not a particular target sequence may have been present in the sample). The data obtained from an array reading (processed or not) may be forwarded (such as by communication) to a remote location if desired, and received there for further use (such as further processing). Stated otherwise, in certain variations, the subject methods may include a step of transmitting data from at least one of the detecting and deriving steps, to a remote location. The data may be transmitted to the remote location for further evaluation and/or use. Any convenient telecommunications means may be employed for transmitting the data, e.g., facsimile, modem, internet, etc. Data may represent a floating point number or integer, for example.

A set of digital signals for a pixel (or an analog signal represented thereby) may be “saturated”, “partially-saturated” or “non-saturated” depending on the number of saturated digital signals within the set. The digital signals in a saturated set of digital signals are all saturated, none of the digital signals in a non-saturated set of digital signals are saturated, and some but not all of the digital signals within a partially-saturated set of digital signals are saturated. Saturated digital signals may be identified by virtue of the fact that they are at maximal magnitude, and non-saturated digital signals may be identified by virtue of the fact that they are below maximal magnitude;

By “area” of an array is meant a region that is the subject of detection by the subject multi-detector system. An area of an array may be as small as a single pixel or as broad as one or more features, dependent upon how the system is configured. In one embodiment, an area corresponds to the dimensions of a pixel.

The term “sensitivity” when used in describing a detector refers to the detector's ability to detect light of a given intensity, with high sensitivity detectors being able to detect low intensity light, where such detectors may be saturated at higher light intensities) and low sensitivity detectors being to detect higher intensity light without saturation. By light intensity is meant the amount of energy, e.g., in the form of photons, per unit area in a signal, e.g., detected at a detector.

By “dynamic range” is meant the range of light signal intensity that can be detected by a detector, without signal saturation or production of a signal that is not significantly above background. By signal intensity is meant the amount of energy, e.g., in the form of photons, per unit area in a signal, e.g., detected at a detector.

A “predetermined wavelength of light” is detectable light of a particular wavelength emitted by a label that indicates the presence of the label. A particular wavelength of light may contain a range of wavelengths (e.g. ±5 nm or more) that contains the wavelength at which emission of the label is at a maximum. While the detected wavelength of light may vary, in representative embodiments it ranges from about 400 to about 800 nm, such as from about 550 to about 610 nm and including from about 650 to about 750 nm. Wavelengths of particular interest include, but are not limited to, the emission maxima of the following fluorescent labels: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g., Cy3, Cy5 and Cy7 dyes; coumarins, e.g. umbelliferone; benzimide dyes, e.g. Hoechst 33258; phenanthridine dyes, e.g., Texas Red; ethidium dyes; acridine dyes; carbazole dyes; phenoxazine dyes; porphyrin dyes; polymethine dyes; BODIPY dyes and quinoline dyes.

Although the system has been described with reference to detecting a single predetermined wavelength light (e.g., light from a single fluorescent label), also encompassed by the invention are systems and methods detect two or more pre-determined wavelengths of light, for example, “red” and “green” light emitted from fluorescent cyanine dyes that separately serve as an input light. Accordingly, in certain cases, a reader may contain two multi-detector systems, each system for detecting a different pre-determined wavelength of light. Pairs of compatible pre-determined wavelengths of light include, but are not limited to, those emitted by: Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPYV-1002 and BODIPY V1005 (Molecular Probes, Eugene, Oreg.), POPO-3 and TOTO-3 (Molecular Probes, Eugene, Oreg.), POPRO3 and TOPRO3 (Molecular Probes, Eugene, Oreg.), Pyrene, Coumarin, Diethylaminocoumarin, Fluorescein Chlorotriazinyl, Fluorescein, R110, Eosin, R6G, Tetramethylrhodamine, Lissamine, Napthofluorescein, Napthofluorescein, etc. Further suitable distinguishable detectable labels may be found in Kricka et al. (Ann Clin Biochem. 39:114-29, 2002).

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “providing” encompasses such terms as “generating”, “identifying” and “producing”.

### DETAILED DESCRIPTION

Methods and systems for reading chemical arrays are provided. Aspects of the methods include reading an array at a first and second detector gain setting to produce two data sets, e.g., in the form of images, of an array, where each reading may be made using a different detector gain setting. Aspects of the methods further include extracting features from the produced data sets, where in certain embodiments a merged image feature extraction protocol is employed. Aspects of the invention further include programming for operating devices, e.g., chemical array readers.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As summarized above, embodiments of the invention provide methods of reading a chemical array. The methods of the invention may be employed with a variety of different chemical array readers, where chemical array readers employed in certain embodiments are further described in greater detail below. Aspects of the methods include positioning a chemical array in a reading position, e.g., a slot of an array carousel, of a chemical array reader and reading the positioned array at least twice without moving the array from the reading position. As such, the array is read in the reading position of the array reader a first time and then, before moving the array from the reading position, the array is read at least one additional time. The number of times the array may be read before moving the array out of the reading position may vary, such as two or more, three or more, four or more, etc. In certain embodiments, the array is read twice, such that the array is read a first time and then re-read before moving the array out of the reading position. The time interval between any two readings of the array may vary, and in certain embodiments the array is immediately read a second time after the first reading. By “immediately” is meant that the interval between the first and second reading does not exceed 120 seconds, such as does not exceed 30 seconds, including does not exceed 1 second, and in certain embodiments is less than 1 second.

In certain embodiments, a different detector gain is employed for at least two of the different readings. For example, in certain embodiments where an array is read a first time and a second time, the array is read the first time with the array detector set at a first gain and the array is then read the second time with the array detector set a second gain. The term “gain” refers to the amount by which an input signal is amplified by the detector. By modulating the gain of a detector an operator can modulate the sensitivity of that detector to emitted light. Aspects of the invention include using a second detector gain setting that is less than the first detector gain setting. In certain embodiments, the second detector gain setting is 1.5× or less than the first detector gain setting, such as 5× or less than the first detector gain setting, including 50× or less than the first detector gain setting. The specific gain settings employed will vary depending on the type of detector and the desired difference between the two settings. In one embodiment, the first detector gain setting is more sensitive such that the detector has a dynamic range that allows it to distinguish weak light signals from background signals. The second detector gain setting is less sensitive and may be set to have a dynamic range that allows it to produce a non-saturated signal when detecting high intensity light. By using both settings to produce linked images, the overall effective dynamic range of the reader may be increased and a more accurate reading of an array, as compared to a reader containing a single detector using a single gain setting, may be obtained. Where the detector is a photomultiplier tube (PMT), an illustrative first detector gain setting may range from 400V to 600 V (or 40-60 counts per chromophore per square micron) and the second detector gain setting will be a desired percentage, e.g., 10%, 25%, etc., of the first detector gain setting. Where an array is read a first time at a first detector gain setting and then at a second time at a second detector gain setting (where the neither the first or second detector gain settings are the “off” state of the scanner but in fact “on” settings where the detector produces a signal in response to light), the array is considered to have been read “in an extended dynamic range” mode, i.e., in an XDR mode.

In certain embodiments, prior to the above reading step in which an array is read two or more times without moving the array from the reading position, a selection is made, e.g., by a user, whether to read an array in an XDR mode. The selection may be a simple “yes” or “no” decision. For example, in certain embodiments following a decision to read in XDR mode, the reader may employ default settings for the first and second detector gain settings. In yet other embodiments, the user may further select the first and second detector gain settings, e.g., from a menu of choices or by manual input. The selection may be made using any convenient protocol, e.g., via a graphical user interface (GUI), for example in the form of a pull down menu, a selection box, etc. The XDR selection can be made for multiple different arrays (e.g., all the arrays present in a carousel), for subset of arrays of a collection of arrays (e.g., for an individual or small number of the total number of arrays of a carousel), for a given color channel of the device, etc. The decision may be made by a user or a machine, e.g., computer, such as a computer programmed to employ a decision tree to arrive at a decision.

Reading an array as described above two or more times produces two or more data sets, e.g., in the form of images, such as tiff images, of the array. Each data set includes data concerning signals generated as a function of location on the array surface. In certain embodiments, the produced data sets are linked, e.g., using an identifier that can be recognized, e.g., by a processor, by a user. An example of such an identifier is a name identifier, where a common core name is employed with a variation for each image, e.g., “Array#1scan_high” and “Array#2scan_low.” In another embodiment, a method that embeds an ID in the tiff header (e.g., that a user cannot easily modify, is employed. For example, a globally unique identifier can be generated and embedded into the description field of the tiff. The description field is not easily modifiable but still visible if the user wishes to find tiff images manually that link together. The GUID is prefaced with XDR ID=“ and ended with ”. This scheme makes it easy for software to look to see if the tiff is part of a XDR scan or not and to find the GUID needed to be matched.

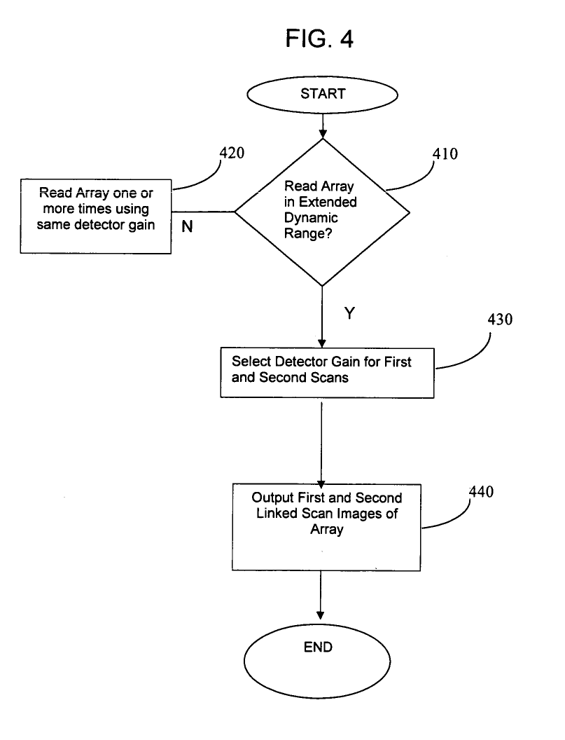

FIG. 4 provides a flow diagram of an array reading protocol according to an embodiment of the invention. In the process depicted in FIG. 4, prior to reading the array a decision is first made at decision box 410 as to whether to read the array in extended dynamic range (XDR) mode or not. The decision may be made by a user or a machine, e.g., computer, such as a computer programmed to employ a decision tree to arrive at a decision. If the answer is no, the array is then read one or more times at a single detector gain setting, as shown in box 420. If the decision is made to read the array in XDR mode, the first and second detector gain settings are chosen, as shown in box 430. This choice may be made by the reader implementing default settings, e.g., 100% and 10%, or input by a user, as described above. The reader then reads the array a first time at the first detector gain setting and a second time at the second detector gain setting to produce first and second data sets of the array (e.g., in the form of first and second scan images), where the data sets are linked, e.g., by a name identifier as described above (as shown in box 440). In another method, the user inputs the second gain setting by selecting the multiple of dynamic range that they want (i.e. in the case of 100% followed by 10% they would have selected first scan at 100% and 10× increase in dynamic range.

Aspects of the invention further include methods of extracting features from product linked array data sets, e.g., as produced by the methods described above. By “extracting features” is meant processing a raw image to obtain a processed signal value for one or more features, where the processed signal value may be a value in which one or more adjustments have been made as compared to an in initial value, e.g., where a background value has been subtracted, where erroneous features have been flagged, e.g., features that do not meet predetermined quality criteria, e.g., due to non-uniformity, image processing, e.g., finding the features in the image and assessing a signal intensity value for that area—and thus linking that to a signal for a given probe, etc.

An aspect of certain embodiments is that data from two or more linked data sets, e.g., images, are combined to produce a merged data set. Combining the data sets may be accomplished in a number of different ways. In certain embodiments, the data sets may be combined by merging the linked images, e.g., by finding the grid locations on that array and actually combining the tiff images. In these embodiments, the tiff images may be combined in a pixel by pixel format following determination of the correct registration between the two images to connect the correct pixels. Then once the tiffs are combined the normal “feature extraction” route could be used. In this embodiment, the tiff combining step would be done such that pixels over a certain level (i.e, saturated or partially saturated pixels, would come from the lower gain scan pixels that are very dim and those near the sensitivity limit would come from the higher gain scan. In another embodiment, the data sets may be merged by image processing the two tiffs separately. For example, a feature signal intensity for each feature may be determined. Following desired further data processing (e.g., background subtraction) the background subtracted signals may then be combined, followied by completion of the rest of the desired data analysis steps, e.g., those normally used in a Feature Extraction protocol of a single tiff image. When the two or more linked data sets are obtained using different detector gain settings, e.g., as described above, the merged data set represents an effective extension of the dynamic range of the chemical array reader, by which is meant that the dynamic range of signals present in the merged data set is wider than is present in a single data set taken with a single detector gain setting.

As such, aspects of the invention include the production, for each label employed in an array assay, of two (or more) linked data sets. In certain embodiments, the linked data sets, e.g., images, may be combined to produce a final data set that contains data that accurately reflects (i.e., describes) the actual amount of light emitted by the surface of the array as compared to either of the two data sets individually. The data sets are combinable using a variety of methods. Embodiments illustrating several of many straightforward methods that could be employed are set forth below. In these embodiments, the data sets contain a numerical value representing the intensity of each pixel of a scan.

In one embodiment, the data set produced by the first detector gain setting may be processed to identify pixels having values that are saturated (e.g., 2¹⁶ for a 16-bit scanner. Saturated values may be substituted by the values for the same pixel or feature depending the particular combination protocol employed (e.g., whether it is a pixel based or feature based protocol) produced by the second detector gain setting to produce the final “merged” data set. In an alternative embodiment, the data set produced by the second detector gain setting may be processed to identify pixels or features having values that are not significantly different from background. Such values may be substituted by the values for the same pixel feature produced by the first detector gain setting to produce the final data set.

In a further embodiment, a processor may first identify data in either data set that is indicative of saturation and data that is indicative of being below the sensitivity of the given detector, and thereby determine useable data. Non-saturated signals may be detected because they are not at maximal magnitude. Additionally, signals indicating the presence of a detected label may be distinguished from background noise by virtue of the fact that the signal is above a set minimum point.

In certain embodiments, a predetermined threshold may be employed which is used to determine those features that should use the lower gain and those features that should use the higher gain. In certain embodiments, a check may be run such that on either side of the predetermined threshold the data from the lower and higher gain settings are linearly related by the same factor, e.g., where the second scan is 10× dimmer than the first scan. This proportionality will stop being the same when the scans are near their detection limit or their saturation limit. For instance, if scan 1 is the high gain scan and scan 2 is the low gain scan, the proportionality of signals within the threshold from scan 1/scan 2 is 10. When scan 1 starts to saturate, then the proportion of signals of the scans will start to drop below 10 because the signals in scan 2 are still increasing but in scan 1 they are pegged at saturation. This proportion will start to drop as soon as the scan 1 becomes partially saturated. At the dim end, when scan 2 hits the background it will stop decreasing its signal (the features will peg at the background noise) and the signals from scan 1 will keep decreasing. As such, the proportion will start to drop below 10×. A threshold may be chosen for signals above which use scan 2 is employed and below which scan 1 is employed. Then this threshold can be checked to make sure that it is well within the region where the proportion is 10 and that it is not in the range where the proportion has changed from 10 significantly.

Once useable data has been determined, the processor may then identify the useable data in each data set that refers to the same pixel and then compare the data referring to that pixel in the first set with the data referring to that same pixel in the second set. For example, the processor may compare individual data sets representing pixels derived from the outputs of the two detector gain settings and select data in either set that is non-saturated, within the sensitivity of a given detector (i.e., the data is distinguishable from background noise), and has the highest relative magnitude (i.e., signal strength). In other words, if a plurality of data sets of unsaturated input signals for a given pixel are detected, each set point for a given pixel from both data sets can be compared and the data that has the greatest overall magnitude for a given pixel may be selected to produce a numerical evaluation of the pixel which may then be output a single, integrated signal.

In another embodiment, each of the data sets may be subjected to feature extraction, to produce a numerical value for each feature, where the numerical value is correlatable with the intensity of signal at each feature. In the same manner as described above, values for saturated features produced by the high sensitivity detector may be substituted by the values for the same features produced by the low sensitivity detector to produce a final data set. Likewise, values for saturated features produced by the low sensitivity detector may be substituted by the values for the same features produced by the high sensitivity detector to produce a final data set.

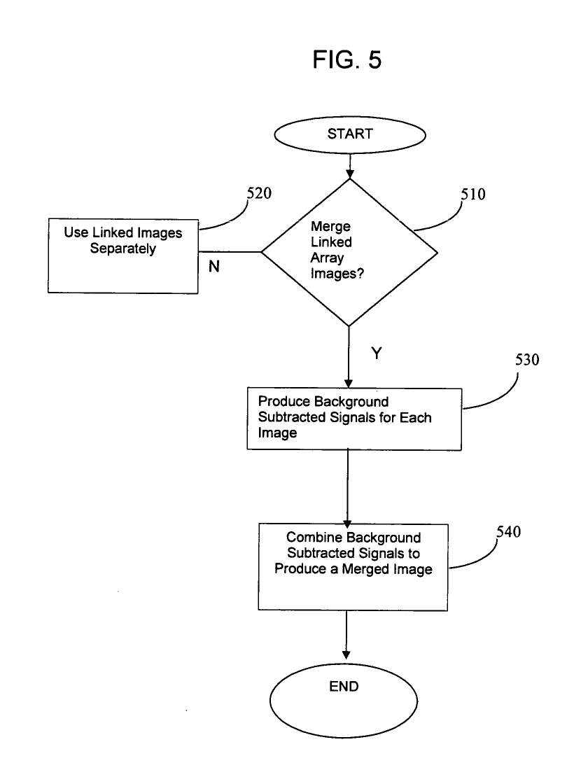

A flow diagram of one embodiment of a feature extraction method of the invention is provided in FIG. 5. In FIG. 5, a decision is first made, e.g., by a user via a graphical user interface or a computer, to use a set of two or more linked images separately (as represented by box 520) or to merge the linked image into a merged image as shown by decision box 510. If the images are to be used separately, each image may be separately subjected to a desired feature extraction protocol for further use. In this decision step (performed by a user or a computer), one may employ a feature extraction program that recognizes pairs of linked images, e.g., produced using the method shown in FIG. 4 or produced using a two detector system, such as disclosed in co-pending application Ser. No. 11/290,100 filed on Nov. 29, 2005, the disclosure of which is herein incorporated by reference) and upon recognition of linked images, provides a choice to a user of whether to use the images separately or merge the images.

If the linked images are to be merged, in the embodiment shown in FIG. 5, the images are first subjected to a background signal subtraction step (box 530), where a background value is subtracted from detected signals for each feature of each image, such that the signals of the linked images are converted to background subtracted signals. This step produces background subtracted signals for each of the features of the images. Any convenient protocol may be employed to convert signals to background subtracted signals, where suitable protocols include those described in Published U.S. Patent Application Nos. 20020051973 and 20020068293; the disclosures of which applications are herein incorporated by reference.

Following conversion of the signals of the linked images to background subtracted signals, the background subtracted signals are then combined into a merged set of signals, e.g., in the form of a merged image, as shown in Box 540.

In certain embodiments, prior to the production of a merged set of background subtracted signals, the background signals of a second of the linked images of a pair are corrected to account for differences in detector gain used to generate the two images. By “corrected to account for differences in detector gain” is meant that the magnitudes of a background-subtracted signals of a second image are adjusted, e.g., multiplied by a factor, referred to herein as a “detector gain difference correction factor,” which accounts for the difference, e.g., lower, detector gain employed in the second reading, as compared to the first reading.

The detector gain difference correction factor may be determined using a number of different protocols. In one embodiment, the detector gain difference correction factor is the same as the predicted experimental correction factor (based on the input for scanner gain). The predicted experimental correction factor is a correction factor that is based solely on the difference of the detector gain settings between the first and second read. For example, if the first detector gain setting is 100% and the second detector gain setting is 10%, then the predicted experimental correction factor would be 10×, such that the signals obtained in the second read would be multiplied by a factor of 10 so as to be corrected for difference in detector gain between the first and second reads.

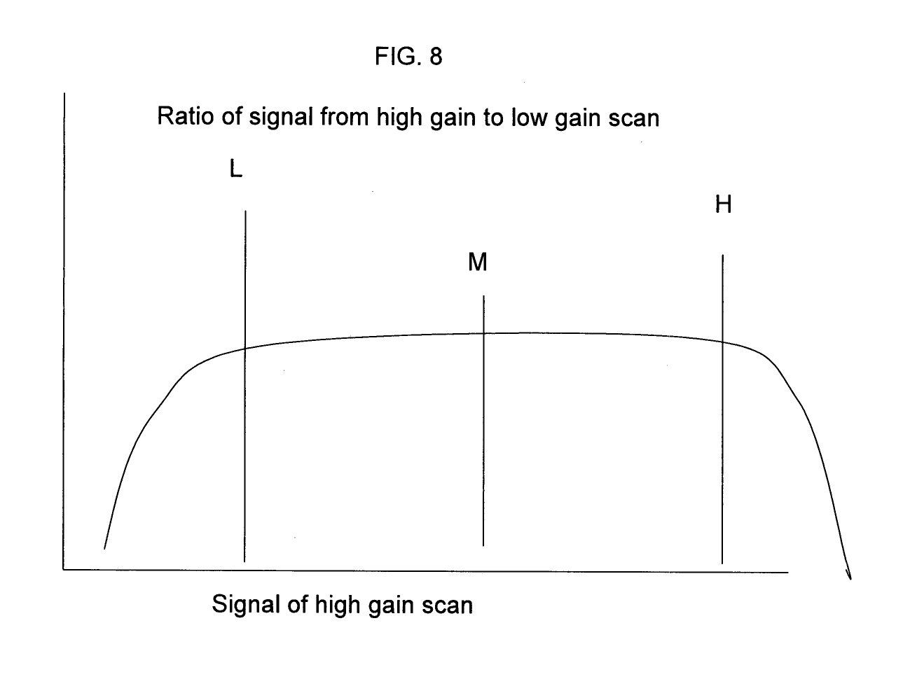

In yet another embodiment, the detector gain difference correction factor is the median of the ratios of the actual differences between corresponding signals in the first and second linked images. In these embodiments, an acceptable signal range is first determined in which all of the signals from the first and second images are predicted to fall, such that a desired portion of, such as a majority of, including all of, the data from the first and second images overlaps and is present in the acceptable signal range. The acceptable signal range is defined by a selected upper threshold (H) and lower threshold (L), where the upper and lower thresholds may be selected by a user or be default values, as desired. As described above, this H and L could be the limits of the signal range where the proportion between scan 1 and scan 2 are roughly constant, see e.g., FIG. 8. Between L and H the ratio between the scans (i.e., the high and low gain scans) is roughly constant. A threshold M may be selected to choose where we use the scan 1 data and scan 2 data. Here L and H could be determined by allowing the ratio to change by a certain % from its amount on the flat region or simply at M. Alternatively, L, M and H might be predetermined.

After the acceptable signal range is defined, the ratios of the signals for at least some of the features, including all the features, from the linked images are determined. For example, the ratio of the magnitudes of the background—subtracted signals for features 1 to n in the first and second linked images are determined, such that one obtains a ratio (e.g., determinined manually or by a computer) for each feature of 1 to n and thereby obtains a distribution of ratios. In certain embodiments, ratio determination is done by suitable computer programming (also provided by the invention as described below) that communicates with the reader. The median of this distribution of determined signal ratios is then employed as the detector gain difference correction factor. As such, this step of this embodiment includes determining the median of the ratios of the signals from the linked images that fall within the acceptable signal range to determine the detector gain difference correction factor.

In certain embodiments, the distribution of determined ratios may be employed to detect potential errors, e.g., in the generation of one or more of the linked images. For example, in certain embodiments the width of the distribution of the determined ratios is employed as a quality control check. In such embodiments, where the width (e.g., standard deviation or (IQR)) is more than a predetermined value, e.g., 1% or 10% or 100% etc., of the identified median, there may be a problem with one scan and/or the other, and the user may be alerted to such problem. Alternatively or in addition, the median is compared to the predicted experimental correction factor. If the difference in factors is greater than a predetermined threshold (e.g., they are different by more than 1% or 10% or 100%), such a difference may indicate a problem and the user may be alerted to such problem. An alert could then be employed by a user to stop the extraction, or proceed knowing that there may be a problem with the extraction.

In certain embodiments, any error term calculated in the background subtracted signal level of the second image may also be multiplied by the desired detector gain difference correction factor. If the width of the ratio is small compared to the multiplicative noise term, then this might be the only error propagation necessary. For example, for each scan an additive (background) noise term (A) and a multiplicative (or proportional to the signal) noise term (M) may be calculated. The additive noise may represent the uncertainty of the background and hence very dim signals. The proportional noise term increases as a fixed multiple of the signal. These terms may be calculated separately on each scan on the background subtracted signal. When scan 2 is converted to the same signal range as scan 1 (by multiplying by a correction factor (e.g., 10×)) these noise terms may also be scaled up. As such, in certain embodiments when the signal is scaled up, the background noise may be scaled up by 10× and the multiplicative noise would scale up automatically because the signal has increased. In certain embodiments, there may be uncertainty in how much to multiply scan 2 by, e.g., where 10× may be selected but 9× or 11× would be more accurate. In such embodiments where there is uncertainty about this factor, the factor may be propagated to the other noise terms and increase the noise term of scan 2 after it is scaled. For example, the uncertainty may be denoted as F. F can be estimated as the width of the distribution of ratios between scan1 and scan2 between the signals of L and H divided by the median. If F is very small (e.g.,1%) compared to M (e.g., 9%) then there is no large effect from propagating the errors. This is because the errors would be propagated in quadrature New Noise=sqrt (M̂2+F̂2), so if F is small compared to M then the New Noise is ˜M. If F approaches M (lets say that F̂2 is more than 1% or 10% or 50% of M̂2) then the user could be warned of the error propagation used. And if F̂2 is small then a simple error propagation (i.e. ignore F) may be employed without warning the user.

Following correction of the signals from the second image, e.g., as described above, a merged image (or other form of merged data set) is prepared. In certain embodiments, the merged image is one in which signals from saturated features from a first of the linked images are substituted by signals from corresponding features from a second of the linked images. For example, a threshold value can be determined, where signals in the first scan that are above the threshold value are substituted with signals from the second scan. As such, where a threshold value might be 30,000 average counts/pixel in a feature, the detector gain difference corrected signals for those features of the second image are employed for those features that exceed this threshold value in the first image. In yet other embodiments, rather than selected the data from scan1 or scan 2, between L and H, the data used could be the average of scan 1 and scan2 (unless one data point is flagged as an outlier (e.g., due to non-uniformity, etc.)

As such, in certain embodiments the data sets of the linked images include one or more “bright” pixels that are saturated in the data set produced using the first detector gain setting and not saturated in the data set produced using the second detector gain setting. If this is the case, a merged image can be produced in which the data point(s) produced using the second detector gain setting corresponding to the saturated data points obtained using the first detector gain setting may be selected for inclusion in the final data set. Likewise, a data point for a “dim” pixel (i.e., a pixel that has a low signal) may not be above background in the data set produced using the second detector gain setting and significantly above background in the data set produced using the first detector gain setting. In this case, the data point produced using the first detector gain setting may be selected for inclusion in the final or merged data set, e.g., in the final image. In this manner, the subject system may be employed to effectively increase the dynamic range of an array reader by reading an array using two detector gain settings, and combining the data produced using the detectors.

Following production of the merged image, e.g., as described above, the merged image may then be subjected to further feature extraction processing, e.g., dye normalization, flagging of erroneous features, determining confidence interval, etc., as desired.

The merged image produced, e.g., as described above, represents an effective extension of the dynamic range of the chemical array reader, i.e., an extended dynamic range (XDR). A reader that is operated in an XDR mode, e.g., as described above, is both more sensitive to light signals of lower intensity, allowing it to better distinguish positive signals from background noise, and additionally less sensitive to light signals of higher intensity, allowing it to detect light signals of higher intensity without becoming saturated. Hence, by using the teachings of the subject invention, the dynamic range of a reader may be increased by at least 10-fold, 100-fold, 1×10³-fold, 1×10⁴-fold, 1×10⁵-fold, 1×10⁶-fold, 1×10⁷-fold, 1×10⁸-fold or more relative to a reader that reads in a non-XDR mode, e.g., at a single detector gain setting. A reader operated in XDR mode may have a dynamic range from of at least about 1 to about 1×10⁵, at least about 1 to at least about 1×10⁶, at least about 1 to at least about 1×10⁷ at least about 1 to at least about 1×10⁸ or more, depending on the exact components used.

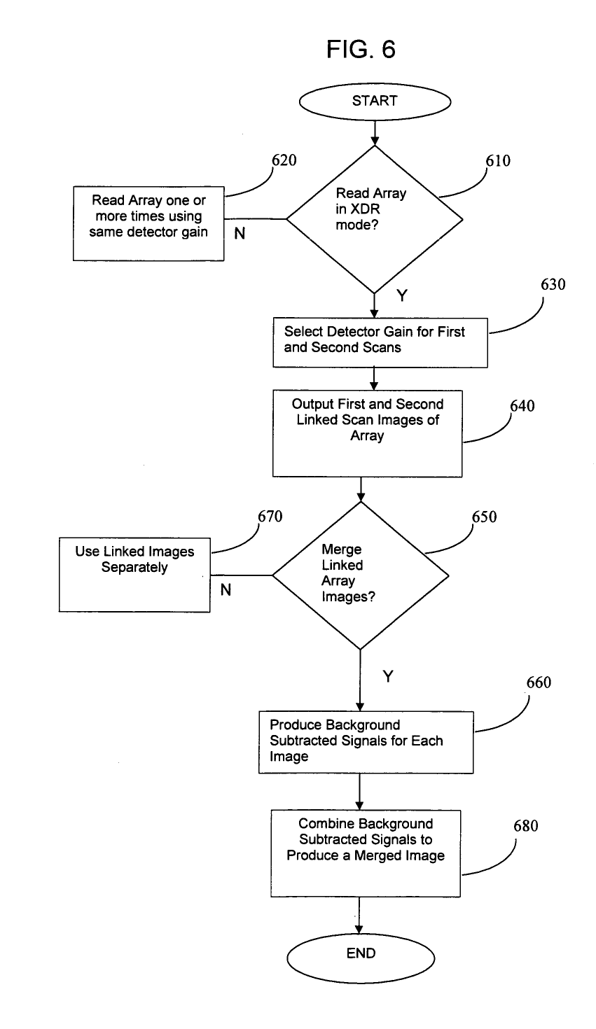

An XDR protocol according to an embodiment of the invention depicted in the flow chart of FIG. 6. In the process shown in FIG. 6, when a decision (made by a user or a computer) is made at decision box 610 to read an array in XDR mode, for a single wavelength of light (e.g., a single “channel”, e.g., a “green” or “red” channel), at least two sets of data, e.g., images, are obtained from a reading of an array: a first set of data set obtained from the detector at a first gain setting and a second set of data set obtained from the detector at a second gain setting, as shown by boxes 630 and 640. Next, a decision made, e.g., by a user via a graphical user interface, to use a set of two or more linked data sets (e.g., images) separately (as represented by box 670) or to merge the linked data sets into a merged data set or image, as shown by decision box 650. If the linked images are to be merged, in the embodiment shown in FIG. 6, the images are first subjected to a background signal subtraction step (box 660), where a background value is subtracted from detected signals for each feature of each image, such that the signals of the linked images are converted to background-subtracted signals. This step produces background-subtracted signals for each of the features of the images. Following conversion of the signals of the linked images to background subtracted signals, the background-subtracted signals are then combined into a merged set of signals, e.g., in the form of a merged image, as shown in Box 680.

The invention also provides a variety of computer-related embodiments. Specifically, the methods described may be executed by a processor in accordance with instructions from a computer program product. In certain embodiments, the above methods are coded onto a computer-readable medium in the form of “programming” or “programming products” as instructions, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer. Therefore, the computer program product comprises programming coded onto computer-readable medium, and the programming and the processor may be part of a computer-based system.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming, or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

The subject systems and methods find use in chemical array readers. Accordingly, aspects of the invention include array readers programmed to read a chemical array, e.g., as described above. Such readers may have a laser excitation system for emitting light from the surface of an array, hardware for performing the methods described above, and, usually, a storage medium for storing data produced by scanning. A reader may also contain or communicate with a processor including programming for executing the subject methods. Array readers may measure a single wavelength of light or measure at least two, and sometimes three, four or five or more wavelengths of light from the surface of an array. In certain embodiments, a subject readers are configured to read in two channels, e.g., corresponding to the “red” and “green” channels of light emitted in typical array experiments (Cheung et al., Nature Genetics 1999, 21: 15-19).

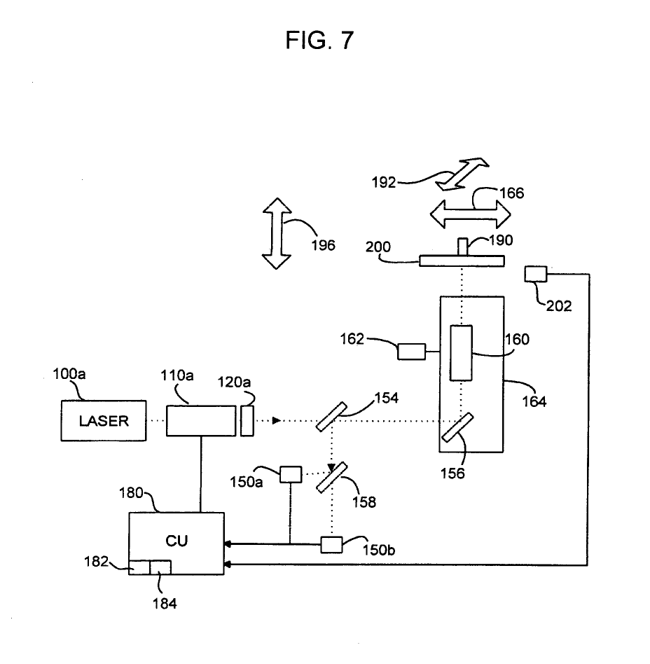

Any optical reader or device may be provided to include the above programming. Representative optical readers of interest include those described in U.S. Pat. Nos: 5,585,639; 5,760,951; 5,763,870; 6,084,991; 6,222,664; 6,284,465; 6,329,196; 6,371,370 and 6,406,849—the disclosures of which are herein incorporated by reference. An exemplary optical reader as may be used in the present invention is shown in FIG. 7.

In FIG. 7, an exemplary apparatus of the present invention (which may be generally referenced as an “array reader”) is illustrated. A light system provides light from laser 100a which can be regulated to control the optical power arriving at the array. In this schematic illustration in FIG. 7, the laser is regulated via an external electro-optic modulator (EOM) 110a with attached polarizer 120a. A second laser (not shown) may be included, wherein each laser would emit a different wavelength of light (e.g., one providing red light and the other green light) and each would have its own corresponding EOM and polarizer. A control signal in the form of a variable voltage applied to the EOM 110a by the controller (CU) 180, changes the polarization of the exiting light which is thus more or less attenuated by the corresponding polarizer 120a. Controller 180 may be or include a suitably programmed processor.

Thus, the EOM 110a and corresponding polarizer 120a together act as a variable optical attenuator which can alter the power of an interrogating light spot exiting from the attenuator. This function can also be performed using any number of variable attenuators, including liquid crystal-based modulators and variable neutral density filters. Alternatively, some lasers can be modulated directly, via analog control signals. The remainder of the light from laser 100a is transmitted through a dichroic beam splitter 154, reflected off fully reflecting mirror 156 and focused onto an array mounted on holder 200, using optical components in beam focuser 160. Light emitted (in particular, fluorescence) at a predetermined wavelength (e.g., green or red light) from features on the array, in response to the interrogating light, is imaged using the same optics in focuser/scanner 160, and is reflected off a mirror 156 and dichroic beamsplitter 154.

The predetermined wavelength of light (e.g., green or red) will then be detected by a detector (e.g., 150a). The detectors employed herein may be any instrument capable of capturing an optical emission of energy (e.g., photons) and converting that energy into an analog and/or digital signal. For instance, one or more of the detectors may be a member of the group including a photo-multiplier tube (PMT), a photodiode (PD), a silicon photodiode (SiPD), an avalanche photodiode (APD), a charge-coupled device (CCD), a charge-injection device (CID), a complementary-metal-oxide-semiconductor detector (CMOS) device, a visible light photon counter, or the like.

All of the optical components through which light emitted from an array or calibration member in response to the illuminating laser light, passes to detector 150a, together with those detectors, form a detection system. This detection system has a fixed focal plane. A scan system causes the illuminating region in the form of a light spot from a laser 100a and a detecting region of detector 150a (which detecting region will form a pixel in the detected image), to be scanned across multiple regions of an array or array package mounted on holder 200. The scanned regions for an array will include at least the multiple features of the array. In certain embodiments, the scanning system is a line by line scanner, scanning the interrogating light in a line across an array when at the reading position, in a direction of arrow 166, then moving (“transitioning”) the interrogating light in a direction into/out of the paper as viewed in FIG. 7 to a position at an end of a next line, and repeating the line scanning and transitioning until the entire array has been scanned.

This scanning feature is accomplished by providing a housing containing mirror 156 and focuser 160, which housing can be moved along a line of pixels (i.e., from left to right or the reverse as viewed in FIG. 7) by a transporter 162. The second direction 192 of scanning (line transitioning) can be provided by second transporter which may include a motor and belt (not shown) to move caddy 200 along one or more tracks. The second transporter may use a same or different actuator components to accomplish coarse (a larger number of lines) movement and finer movement (a smaller number of lines). Generally, directly adjacent rows are scanned. However, “adjacent” rows may include alternating rows or rows where more than one intervening row is skipped.

The optical reader of FIG. 7 may further include a reader (not shown) which reads an identifier from an array package. When identifier is in the form of a bar code, that reader may be a suitable bar code reader.

Of course, the movements 166 and 192 may be accomplished by actuating holder 200 or housing alone. Still further, the movement roles described for each element above may be swapped.

The system may also include detector 202, processor 180, and a motorized or servo-controlled adjuster 190 to move holder 200 in the direction of arrow 196 to establish correct focus for the system. In addition, such an autofocus system may contain a position detector e.g. a quadrature position encoder, also feeding back to the CU measures the absolute position (i.e., relative to the apparatus) of the servo-controlled adjuster 190. As above with respect to movements 166 and 192, focus servo control movement 196 may occur in connection with housing 164 instead of the holder, or, if the detection system is not a fixed focal plane system, by an adjustment of laser focuser 160. Further details regarding suitable chemical array autofocus hardware is described in U.S. Pat. No. 6,486,457, as well as European publication EP 1091229 published Apr. 11, 2001.

Controller 180 of the apparatus is connected to receive signals from detector 150a that are signals which result from the detection of the predetermined wavelength from emitted light for each scanned region of an array the signal from autofocus detector 202, which is used to control the scan system. Controller 180 contains all the necessary software to detect signals from detector 150a and regulate a motorized or servo-controlled adjuster 190 through a control loop. Controller 180 may also analyze, store, and/or output data relating to emitted signals received from detectors 150a in a known manner.

Controller 180 also includes a programmable digital signal processor for performing the methods described above, and may, include a plurality of analog-to-digital converters, and other components of a multi-detector detection system (such as a multi-detector photodetection system), e.g., a current-to-voltage converter, voltage amplifier, etc., as desired, a media reader 182 which can read a portable removable media (such as a magnetic or optical disk), and a communication module 184 which can communicate over a communication channel (such as a network, for example the internet or a telephone network) with a remote site (such as a database at which information relating to array package may be stored in association with the identification).

In one mode of operation, an array in a package is exposed to a liquid sample. This liquid sample may be placed directly on the array or introduced into a chamber through a septa in the housing of the array. After a time to allow, for example, hybridization, the array may then be washed and scanned with a liquid (such as a buffer solution) present in the chamber and in contact with the array, or it may be dried following washing. After mounting a given array in cradle 200 (either with the array features on the glass surface nearer to, or further from, the lens—depending, at least, upon the lens setup) the identifier reader may automatically (or upon operator command) read an identifier from the array package, which may be used to e.g. retrieve information on the array layout from a database containing the identifier in association with such information. Such a database may be a local database accessible by controller 180 (such as may be contained in a portable storage medium in drive 182.

The saved results from a sample exposed array, read with the methods described above, may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication of data representing the results) to a remote location if desired, and received there for further use (such as further processing).

The subject array readers find use in a variety applications, where such applications are generally analyte detection applications in which the presence of a particular analyte in a given sample is detected at least qualitatively, if not quantitatively. Protocols for carrying out array assays are well known to those of skill in the art and need not be described in great detail here. Generally, the sample suspected of comprising the analyte of interest is contacted with an array under conditions sufficient for the analyte to bind to its respective binding pair member that may be present on the array.

Thus, if the analyte of interest is present in the sample, it binds to the array at the site of its complementary binding member and a complex is formed on the array surface. The presence of this binding complex on the array surface is then detected, e.g., through use of a signal production system such as a fluorescent label present on the analyte, etc, where detection includes scanning with an optical reader according to the present invention. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, comparative genomic hybridization (CGH) applications, localization applications, and the like. References describing methods of using arrays in various applications include U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992—the disclosures of which are herein incorporated by reference.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

In using an array in connection with a reader according to the present invention, the array may be exposed to a sample (such as a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. Certain embodiments of the invention may involve transmitting data obtained from a method described above from a first location to a remote location. Certain other embodiments of the invention may involve receiving, from a remote location, data obtained from a method described above.

In reading the array, pixel signals may be processed using the methods described above.

It is further noted that aspects of the invention may be applicable to a variety of optical readers, e.g., scanners, including those that detect chemiluminescent or electroluminescent labels. The present invention will be applicable to such readers where powering down the reader will result in lifetime savings, as exemplified above.

Kits for use in connection with the subject invention may also be provided. In one embodiment, a kit of the subject invention may include a computer program product, e.g., comprising a computer readable medium including programming for operating a reader according to an embodiment of the invention, e.g., for upgrading an existing reader. Additionally, kits of the subject invention usually include at least instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means of upgrading an existing reader. Alternately, the combination may be provided in connection with a new reader in which the software is preloaded on the same. In which case, the instructions will serve as a reference manual (or a part thereof and the computer readable medium as a backup copy to the preloaded utility.

The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g., via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or world wide web. Some form of access security or identification protocol may be used to limit access to those entitled to use the invention. As with the instructions, the means for obtaining the instructions and/or programming is generally recorded on a suitable recording medium.

In addition to the subject programming and instructions, the kits may also include one or more reference arrays, e.g., two or more reference arrays for use in testing an optical reader after software installation.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of reading a chemical array, said method comprising:
(a) positioning said array in a reading position of a chemical array reader;
(b) reading said array a first time in said scan position at a first detector gain setting to produce a first data set; and
(c) before moving said array out of said reading position re-reading said array at a second detector gain setting to produce a second data set.

2. The method according to claim 1, wherein said re-reading occurs immediately after said reading of said array a first time.

3. The method according to claim 2, wherein said method further comprises selecting whether to read said array in an extended dynamic range mode.

4. The method according to claim 3, wherein when a choice is made to read said array in an extended dynamic range mode, said second detector gain setting is less than said first detector gain setting.

5. The method according to claim 4, wherein said method further comprises selecting said first and second detector gain settings.

6. The method according to claim 1, wherein said method further comprises linking said first and second data sets.

7. The method according to claim 6, wherein said first and second data sets are linked by an identifier.

8. The method according to claim 7, wherein said identifier is a name identifier.

9. The method according to claim 1, wherein said method further comprises feature extraction from said first and second scan data sets.

10. The method according to claim 9, wherein said feature extraction comprises merging said first and second data sets into a merged data set.

11. A method of extracting features from two or more linked data sets of a chemical array, said method comprising:
(a) determining whether said linked data sets should be merged or used separately; and
(b) if a decision is made to merge said linked data sets;
(i) converting signals in said linked data sets to background subtracted signals;
(ii) combining said background-subtracted signals into a merged set.

12. The method according to claim 11, wherein prior to said combining step (ii) said method further comprises correcting background subtracted signals to account for differences in detector gain.

13. The method according to claim 12, wherein said correcting comprises adjusting said background subtracted signals from a second of said linked data sets by a detector gain difference correction factor.

14. The method according to claim 13, wherein said detector gain difference correction factor is determined by:
(a) defining an acceptable signal range;
(b) determining the median of the ratios of the signals from said linked data sets that fall within said acceptable signal range to determine the detector gain difference correction factor.

15. The method according to claim 11, wherein said combining comprises selecting data from a given set based on whether a signal in a first set exceeds a threshold.

16. The method according to claim 11, wherein said combining comprises producing a merged set of signals in which signals from saturated features from a first of said linked data sets are substituted by signals from corresponding features from a second of said linked data sets.

17. The method according to claim 11, wherein said combining comprises averaging background subtracted signals from said linked data sets.

18. A computer-readable medium encoding instructions to direct a chemical array reader to perform the method of claim 1.

19. A chemical array reader comprising or communicating with a computer-readable medium according to claim 18.

20. A method of assaying a sample, said method comprising:
(a) contacting said sample with a chemical array of two or more ligands immobilized on a surface of a solid support at different known locations; and
(b) reading said array with a chemical array reader according to claim 19 to obtain two or more linked images of said array.

21. The method according to claim 20, wherein said method further comprises extracting features from said two or more linked images.

22. The method according to claim 21, wherein features are extracted from said two or more linked images using a feature extraction method according to claim 11.

23. The method according to claim 20, wherein said chemical array is chosen from a polypeptide array and a nucleic acid array.

24. A computer-readable medium encoding instructions to direct a computer to perform the method of claim 11.

25. A computer comprising a computer-readable medium according to claim 24.
